(19) United States
(12) Patent Application Publication
(10) Pub. No.: US 2020/0101311 A1
(43) Pub. Date: Apr. 2, 2020

(54) INTRA- AND EXTRA-ORAL PHOTOTHERAPY SYSTEMS AND METHODS

(71) Applicant: COSMOAESTHETICS PTY LTD, KEW, Victoria (AU)

(72) Inventor: Masoud TAHGHIGHI JAFARZADEH, Kew, Victoria (AU)

(21) Appl. No.: 16/616,840

(22) PCT Filed: May 25, 2018
(86) PCT No.: PCT/AU2018/050512

§ 371 Date: Nov. 25, 2019

(30) Foreign Application Priority Data
May 25, 2017 (AU) 2017901988

(51) Int. Cl. A61N 5/06

(52) U.S. Cl. CPC A61N 5/0613; A61N 2005/0632; A61N 2005/0651; A61N 2005/0644; A61N 2005/0647; A61N 2005/0606

(57) ABSTRACT

This disclosure relates to an intra-oral phototherapy device, comprising: a handle; at least one mouthpiece removably connectable to the handle; and at least one light source in the mouthpiece adapted to direct light inside a mouth of a user. It will be apparent that the device described herein has broad applications.

### FIELD

The present invention relates to intra- and extra-oral phototherapy systems and methods.

### BACKGROUND

Conventional solutions for treating or preventing intra- and extra-oral conditions or disorders typically involve surgery and/or drugs. Example intra- and extra-oral conditions or disorders may comprise oral pain, oral infection, oral inflammation, oral trauma, facial swelling, temporomandibular joint (TMJ) pain, headaches, migraine headaches, cluster headaches, head pain, etc.

A need therefore exists for effective, non-invasive and drug-free solutions for treating or preventing intra- and extra-oral conditions or disorders.

### SUMMARY

According to the present invention, there is provided an intra-oral phototherapy device, comprising:

a handle;

at least one mouthpiece removably connectable to the handle; and

at least one light source in the mouthpiece adapted to direct light inside a mouth of a user.

The at least one mouthpiece may be generally half-moon shaped or generally tongue shaped.

The at least one mouthpiece may comprise a plurality of interchangeable mouthpieces.

The at least one mouthpiece may be removably connectable to the handle via a quick-release coupling.

The at least one light source may comprise a plurality of light emitting diodes (LEDs).

The plurality of LEDs may be individually or collectively controllable to vary one or more of wavelength (nm), light output frequency (continuous versus pulsed), power output (mW), power density (W/cm²), energy density (J/cm²), treatment time, total energy delivered (J), and combinations thereof.

The light may have a wavelength between around 300 nm and around 1000 nm, for example, between around 600 nm and around 860 nm.

The present invention further provides a system, comprising:
the intra-oral phototherapy device described above;
the plurality of interchangeable mouthpieces described above; and
a recharging station to recharge the intra-oral phototherapy device and store at least one of the plurality of interchangeable mouthpieces.

The present invention further provides an extra-oral oral phototherapy device, comprising:

a headband with two earcups;

wherein each earcup has an earcup projection comprising at least one light source adapted to direct light at least partially over a temporomandibular joint of a wearer.

Each earcup projection may be adjustably movable relative to each earcup so that the at least one light source is adapted to direct light at least partially over one or more of frontal, temporal, zygomatic, and buccal regions of the wearer's head and face.

Each earcup projection may be adjustably movable relative to each earcup so that the at least one light source is adapted to direct light at least partially over one or more nerves or ganglia associated with one or more of headaches, migraine headaches, cluster headaches, and TMJ pain.

Each earcup projection may be adjustably rotatable relative to each earcup.

The at least one light source may comprise a plurality of LEDs.

The plurality of LEDs may be individually or collectively controllable to vary one or more of wavelength (nm), light output frequency (continuous versus pulsed), power output (mW), power density (W/cm²), energy density (J/cm²), treatment time, total energy delivered (J), and combinations thereof.

The light may have a wavelength between around 300 nm and around 1000 nm, for example, between around 600 nm and around 860 nm.

The present invention also provides a system comprising the intra-oral phototherapy device described above and the extra-oral phototherapy device described above.

The present invention further provides a method of treating or preventing one or more intra- or extra-oral conditions or disorders using one or both of the intra-oral phototherapy device described above and the extra-oral phototherapy device described above.

### BRIEF DESCRIPTION OF DRAWINGS

Embodiments of the invention will now be described by way of example only with reference to the accompanying drawings, in which:

FIG. 1 is a perspective view of an intra-oral phototherapy device with an example interchangeable mouthpiece according to an embodiment of the present invention;

FIG. 2 is a perspective view of the intra-oral phototherapy device with another example interchangeable mouthpiece;

FIG. 3 is a perspective view of the intra-oral phototherapy device provided in a system comprising a charging and mouthpiece storage station; and

FIG. 4 is a perspective view of an extra-oral phototherapy device according to an embodiment of the present invention.

### DESCRIPTION OF EMBODIMENTS

Referring to the drawings, an intra-oral phototherapy device 10 according to an embodiment of the present invention may generally comprise a handle 12 and at least one mouthpiece 14 connectable to the handle 12. At least one light source 16 may be provided in or on the mouthpiece 14 to direct light inside a mouth (not shown) of a user (not shown). The intra-oral device 10 may further comprise a power source (not shown) in the handle 12 to provide power to the at least one light source 16, and a switch 18 on the handle 12 to selectively activate the at least one light source 16.

The handle 12 may be generally cylindrically shaped and at least partially hollow to house the power source. The handle 12 and the at least one mouthpiece 14 may be made as mouldings in medical- or dental-grade plastics. One or both of the handle 12 and the at least one mouthpiece 14 may be provided with medical- or dental-grade hygienic coating or covering (not shown).

The shape and configuration of the at least one mouthpiece 14, and the orientation and field of view of the at least one light source 16, may be selected to direct light inside the mouth generally, or to targeted areas inside the mouth, such as gums, cheeks, roof of the mouth etc.

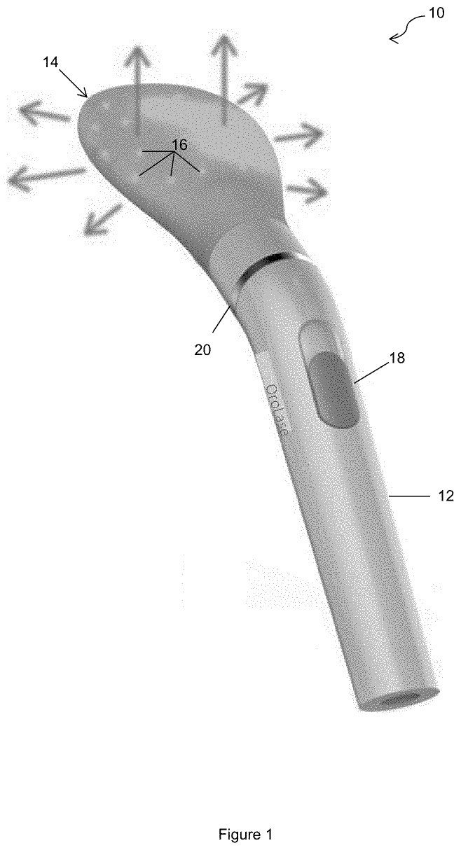

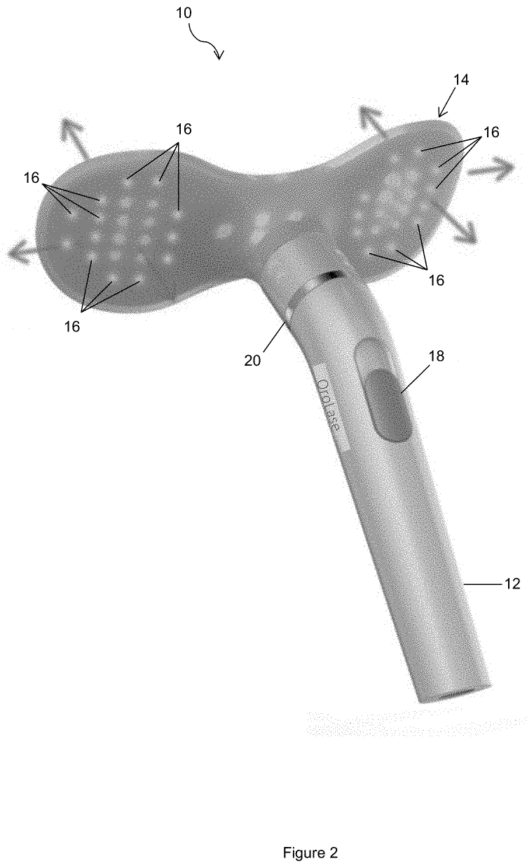

As illustrated in FIGS. 1 to 3, the at least one mouthpiece 14 may comprise a plurality of interchangeable mouthpieces 14. For example, one of the mouthpieces 14 may be generally half-moon shaped as illustrated in FIG. 2, and the other mouthpiece 14 may be generally tongue- or oval-shaped as illustrated in FIG. 1. The at least one mouthpiece 14 may be removably connectable to the handle 12 via a quick-release coupling 20. The quick-release coupling 20 may, for example, comprise a bayonet coupling.

The at least one light source 16 may comprise a plurality of LEDs 16 that are individually or collectively controllable by a LED control circuit (not shown) housed in the handle 12. The LED control circuit may be configured to selectively control one or more parameters of the plurality of LEDs 16 comprising wavelength (nm), light output frequency (continuous versus pulsed), power output (mW), power density (W/cm²), energy density (J/cm²), treatment time, total energy delivered (J), and combinations thereof. The light may have a wavelength between around 300 nm and around 1000 nm, for example, between around 600 nm and around 860 nm.

The power source may comprise a rechargeable battery (not shown). The switch 18 may, for example, comprise a slide switch.

The intra-oral phototherapy device 10 may be used to treat or prevent one or more intra- or extra-oral conditions or disorders. Example intra- and extra-oral conditions or disorders may comprise oral pain, oral infection, oral inflammation, oral trauma, facial swelling, TMJ pain, headaches, migraine headaches, cluster headaches, head pain, etc.

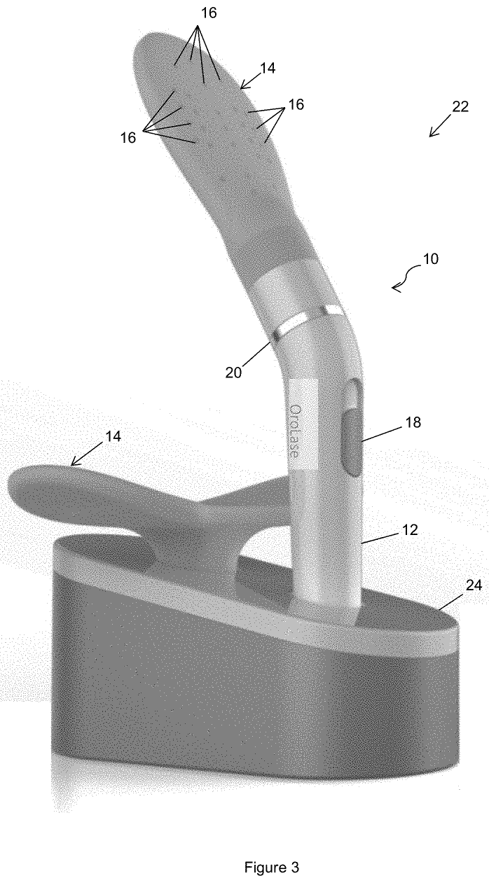

Referring to FIG. 3, in similar fashion to conventional electric toothbrushes, the intra-oral phototherapy device 10 may be provided as a component of a system 22 that comprises the plurality of interchangeable mouthpieces 14, and a recharging station 24. The recharging station 24 may be configured to recharge the intra-oral phototherapy device 10 and store at least one of the plurality of interchangeable mouthpieces 14. The recharging station 24 may, for example, comprise an inductive recharging station.

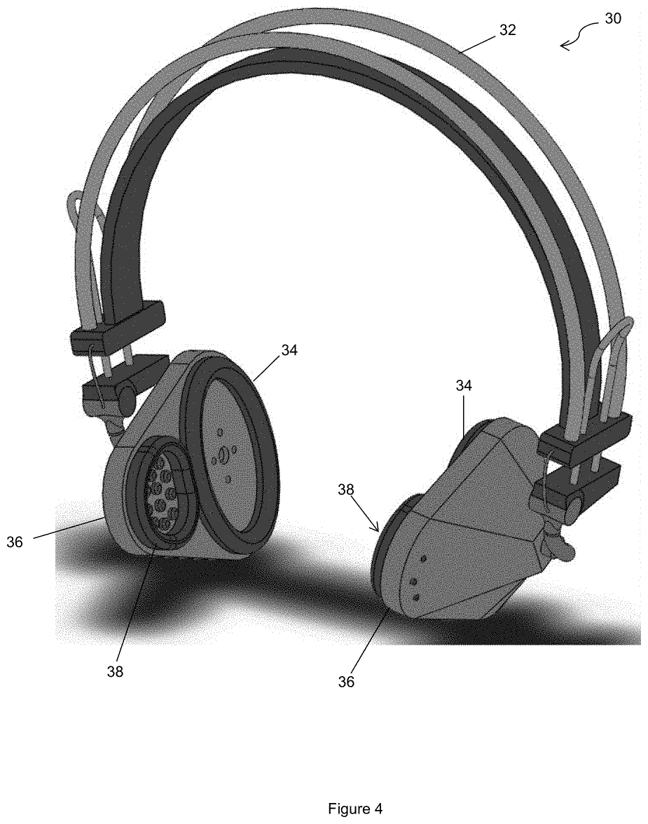

Referring to FIG. 4, an extra-oral phototherapy device 30 according to another embodiment of the present invention may generally comprise a headband 32 with two earcups 34. Each earcup 34 may have an earcup projection 36 comprising at least one light source 38 adapted to direct light at least partially over a temporomandibular joint of a wearer (not shown).

Each earcup projection 36 may be adjustably movable relative to each earcup 34 so that the at least one light source 38 is adapted to direct light at least partially over one or more of frontal, temporal, zygomatic, and buccal regions of the wearer's head and face. Further or alternatively, each earcup projection 36 may be adjustably movable relative to each earcup 34 so that the at least one light source 38 is adapted to direct light at least partially over one or more nerves or ganglia associated with one or more of headaches, migraine headaches, cluster headaches, and TMJ pain. For example, each earcup projection 36 may be adjustably rotatable clockwise or anticlockwise relative to each earcup 34 to direct light over the relevant regions of the wearer's head or face.

The at least one light source 38 may comprise a plurality of LEDs. The plurality of LEDs may be individually or collectively controllable to vary one or more of wavelength (nm), light output frequency (continuous versus pulsed), power output (mW), power density (W/cm²), energy density (J/cm²), treatment time, total energy delivered (J), and combinations thereof.

The light may have a wavelength between around 300 nm and around 1000 nm, for example, between around 600 nm and around 860 nm. For example, the light may have wavelengths of 685 nm and 810 nm when the device 30 is used to treat or prevent cluster headaches and migraine headaches. The light may have wavelengths of 645 nm, 685 nm and 810 nm when the device 30 is used to treat or prevent TMJ pain.

The device 30 may further comprise a power source (not shown), such as one or more rechargeable batteries, provided in one or more of the headband 32 or earcups 34. The device 30 may further comprise a power switch (not shown) and a controller (not shown) configured to provide variable or constant light output frequency. The controller may be further configured to provide a timer to vary the treatment time, for example, in five-minute intervals of 5, 10 and 15 minutes.

The intra-oral phototherapy device 10 and the extra-oral phototherapy device 30 may be used individually or together (ie, in conjunction with one another) to treat or prevent one or more intra- or extra-oral conditions or disorders.

Embodiments of the present invention provide intra- and extra-oral phototherapy systems and methods that are useful for treating, preventing or controlling a wide range of intra-oral or extra-oral conditions or disorders, for example, oral pain, oral infection, oral inflammation, oral trauma, facial swelling, TMJ pain, headaches, migraine headaches, cluster headaches, head pain, etc. Phototherapy is an effective, non-invasive, drug-free solution that has no known side effects. It has been used for reducing pain and speeding healing by stimulating the body's natural repair processes at the cellular level. Embodiments of the present invention offer dental staff and consumers a clinically-proven, drug-free solution to eliminate pain and accelerate soft and hard tissue repair both inside and outside the mouth. For example, embodiments of the present invention may be used to treat, reduce and control:
oral pain and swelling caused by trauma, such as tooth extraction, root canal, oral surgery, etc;
sore throat caused by flu, allergy, illness, etc;
oral infection, Candida albicans, yeast (oral thrush), bad breath, ulceration from dentures or prosthetic restoration, etc;
gum inflammation due to illnesses such as diabetes, AIDS, hepatitis, smoking, heart disease, etc;
creamy white bumps on the tongue, inner cheeks, gums, or tonsils, etc;
improved healing of dry, cracked skin at the corners of the mouth, etc;
difficulty swallowing;
anti-aging, such as reduction of naso-labial folds and cheek plumbing effect due to weight-loss and ageing without the need of dermal filler and injections, etc.

Embodiments of the present invention may also provide the following features and benefits:
collagen stimulation, skin tightening and wrinkle reduction in regions such as naso-labial folds, and upper and lower lip lines;
jowl and cheek skin tightening and volumisation;
anti-inflammatory to treat sore throats, gum disease, sinus pain, and upper respiratory inflammation;
collagen stimulation;
snoring reduction and treatment of sleep apnoea;
antifungal;
hygienic;
can be covered by a single-use plastic shield which can be discarded after each use to avoid cross-contamination and provide oral hygiene control;
non-invasive, no pain, no drugs, no side effects;
can be used on all ages, anyone can use it;
easy to use, easy to carry around, safe, effective, fun, etc;
affordable;
multi-wavelength phototherapy that is clinically proven to treat and control oral related health conditions.

For the purpose of this specification, the word “comprising” means “including but not limited to,” and the word “comprises” has a corresponding meaning.

The above embodiments have been described by way of example only and modifications are possible within the scope of the claims that follow.

1. An intra-oral phototherapy device, comprising:
a handle;
at least one mouthpiece removably connectable to the handle; and
at least one light source in the mouthpiece adapted to direct light inside a mouth of a user.

2. The device of claim 1, wherein the at least one mouthpiece is generally half-moon shaped or generally tongue shaped.

3. The device of claim 1, wherein the at least one mouthpiece comprises a plurality of interchangeable mouthpieces.

4. The device of claim 1, wherein the at least one mouthpiece is removably connectable to the handle via a quick-release coupling.

5. The device of claim 1, wherein the at least one light source comprises a plurality of light emitting diodes (LEDs).

6. The device of claim 5, wherein the plurality of LEDs are individually or collectively controllable to vary one or more of wavelength (nm), light output frequency (continuous versus pulsed), power output (mW), power density (W/cm²), energy density (J/cm²), treatment time, total energy delivered (J), and combinations thereof.

7. The device of claim 1, wherein the light has a wavelength between around 300 nm and around 1000 nm.

8. The device of claim 7, wherein the light has a wavelength between around 600 nm and around 860 nm.

9. A system, comprising:
the intra-oral phototherapy device of claim 3; and
a recharging station to recharge the intra-oral phototherapy device and store at least one of the plurality of interchangeable mouthpieces.

10. An extra-oral oral phototherapy device, comprising:
a headband with two earcups;
wherein each earcup has an earcup projection comprising at least one light source adapted to direct light at least partially over a temporomandibular joint of a wearer.

11. The device of claim 10, wherein each earcup projection is adjustably movable relative to each earcup so that the at least one light source is adapted to direct light at least partially over one or more of frontal, temporal, zygomatic, and buccal regions of the wearer's head and face.

12. The device of claim 10, wherein each earcup projection is adjustably movable relative to each earcup so that the at least one light source is adapted to direct light at least partially over one or more nerves or ganglia associated with one or more of headaches, migraine headaches, cluster headaches, and TMJ pain.

13. The device of claim 10, wherein each earcup projection is adjustably rotatable relative to each earcup.

14. The device of claim 10, wherein the at least one light source comprises a plurality of LEDs.

15. The device of claim 14, wherein the plurality of LEDs are individually or collectively controllable to vary one or more of wavelength (nm), light output frequency (continuous versus pulsed), power output (mW), power density (W/cm²), energy density (J/cm²), treatment time, total energy delivered (J), and combinations thereof.

16. The device of claim 10, wherein the light has a wavelength between around 300 nm and around 1000 nm.

17. The device of claim 16, wherein the light has a wavelength between around 600 nm and around 860 nm.

18. A system comprising the intra-oral phototherapy device of claim 1, and the extra-oral phototherapy device of claim 10.

19. A method of treating or preventing one or more intra- or extra-oral conditions or disorders using one or both of the intra-oral phototherapy device of claim 1, and the extra-oral phototherapy device of claim 10.
